(19) United States
(12) Patent Application Publication
(10) Pub. No.: US 2021/0346710 A1
(43) Pub. Date: Nov. 11, 2021

(54) EEG BASED VARIABLE STIMULATION

(71) Applicants: James William Phillips, Foundtain Valley, CA (US); Robert Isenhart, Orange, CA (US); Alexander Ring, Newport Beach, CA (US)

(72) Inventors: James William Phillips, Foundtain Valley, CA (US); Robert Isenhart, Orange, CA (US); Alexander Ring, Newport Beach, CA (US)

(21) Appl. No.: 16/869,150

(22) Filed: May 7, 2020

(51) Int. Cl. A61N 2/00

(52) U.S. Cl. CPC A61N 2/006

(57) ABSTRACT

Described are methods, and devices for modulating the brain activity of a person by varying pulse interval of repetitive transcranial magnetic stimulation (rTMS) or Transcranial Alternating Current Stimulation (tACS). The pulse interval variation is chosen so that the interval frequency distribution of pulses for a treatment session approximates the EEG frequency distribution of the person.

### FIELD OF THE INVENTION

The present invention relates to methods and devices to modulate brain activity with repetitive transcranial magnetic stimulation (rTMS) or transcranial Alternating Current Stimulation (tACS) wherein the rTMS or tACS pulse interval is variable.

### BACKGROUND OF THE INVENTION

Repetitive Transcranial Magnetic Stimulation (rTMS) and transcranial Alternating Current Stimulation (tACS) have been used to improve symptoms of mental disorders and to modify brain function. rTMS uses high energy magnetic pulses from a magnetic field generator that is positioned close to a person's head, so that the magnetic pulses affect a desired treatment region within the brain. tACS uses electric current pulses delivered to the scalp. Traditionally, the rTMS or tACS pulses are generated at a fixed frequency for a short time duration. For example, a typical rTMS system may generate pulses at 10 Hz for a duration of 6 seconds. A series of pulses generated over a period of time is referred to as a pulse train. An rTMS treatment session may be composed of several pulse trains, with a rest period between each pulse train. A typical rest period may be 54 seconds, such that 6 seconds of rTMS pulses are generated per minute.

The brain's neural oscillations arise from synchronous and coherent electrical activity and can be recorded using an electroencephalogram (EEG). The intrinsic EEG Frequency of a predefined EEG range is the dominant EEG oscillation within that range. For example, the dominant EEG oscillation in the range of 8-13 Hz is the Intrinsic Alpha Frequency (IAF), or simply the alpha frequency, and can vary between individuals and over time. It has been disclosed by Phillips and Jin (U.S. Pat. No. 8,475,354) that providing magnetic pulses at a frequency that matches a person's IAF can provide an added benefit to the person when compared to rTMS at an arbitrary frequency, such as 10 Hz. In addition, it has been disclosed by Jin (U.S. Pat. No. 9,308,385) that rTMS pulses at a harmonic of a non-EEG biological metric, such as heart rate, that is close to the person's IAF may also provide an added benefit.

### SUMMARY

Conventionally, rTMS or tACS pulses are administered at a constant pulse width, amplitude, and/or pulse frequency. However, an individual's EEG may vary over time or may be composed of a variety of individual signals that creates a distribution of attributes, such that additional benefits may be achieved if the applied magnetic field is further customized to the individual EEG signal.

An exemplary embodiment includes applying a magnetic field to a patient where the magnetic field may be varied in amplitude, pulse duration, pulse interval, pulse frequency, pulse train duration, and combinations thereof in response to an analyzed EEG signal of the patient. For example, an EEG recording may vary over time such that the EEG may define a unique pattern or distribution. The unique pattern or distribution may be analyzed to optimize the administration of brain stimulation. The optimal stimulation may be administered at a variable pulse length, variable pulse interval, variable pulse amplitude, variable pulse frequency, variable pulse train duration, and combinations thereof.

As another example, brain activity as shown in EEG recordings, does not occur at a single frequency, but instead is composed of aggregate neuronal firings at a variety of frequencies, such that the frequency spectrum consists of the summations of the rhythmic firings of a large number of neurons around average intrinsic frequencies. Based on this variability in IAF across the brain and over time, brain stimulation, such as that proposed by Phillips and Jin (U.S. Pat. No. 8,475,354) may be optimal in general, but not for each individual region or moment in time. Instead, the optimal stimulation may be administered at a variety of frequencies, such that the frequency distribution of the magnetic field or the induced electric current in the brain approximates the frequency distribution of the recorded EEG of the person at a particular time or time interval.

Described herein are methods and devices to treat a person by varying the pulse duration, pulse interval, pulse amplitude, pulse frequency, and pulse train duration, and any combination thereof of repetitive transcranial magnetic stimulation (rTMS) or Transcranial Alternating Current Stimulation (tACS). The methods and devices described herein do not require any medication. The methods and devices described herein vary one or more attributes of the stimulation, such as pulse duration, pulse interval, pulse intensity, pulse frequency/frequencies, and/or pulse train attributes.

In an exemplary embodiment, the pulse interval of current pulses may be varied so that sequential pulses are administered at a defined time sequence with a variation in the period between pulses. The pulse period may be determined based on a time interval between sequential peaks, sequential troughs, or a combination thereof of a waveform analyzed from a patient's EEG signal. The pulse interval may also be determined based on a variability of frequencies or a distribution of frequencies detected in the EEG signal.

In an exemplary embodiment, the pulse intensity of current pulses may be varied so that the pulses may be administered at an intensity variation based on an amplitude variation of a waveform analyzed from a patient's EEG signal. Sequential pulses may be administered at a variable intensity that is determined or based on an amplitude measured between sequential peaks and troughs or troughs and peak of a waveform generated from an EEG signal of a patient.

In an exemplary embodiment, the frequency of current pulses is varied so that the frequency distribution of current pulses approximates the frequency distribution of an electroencephalogram (EEG) of the person, in order to affect the resonant behavior of neuronal regions in the targeted area that fire with frequencies that are close to the current pulse frequency.

In one aspect of the invention, a method of modulating a brain activity of a person is described wherein said method comprises modulating a brain activity of a person wherein said method comprises subjecting the person to repetitive stimulating current pulses wherein the current pulse interval is variable, and is based on a wave pattern of an EEG profile of the person, and wherein an improvement in a physiological condition or a neuropsychiatric condition is achieved.

In one aspect of the invention, a method of modulating a brain activity of a person is described wherein said method comprises modulating a brain activity of a person wherein said method comprises subjecting the person to repetitive stimulating current pulses wherein the current pulse intensity is variable, and is based on a wave pattern amplitude of an EEG profile of the person, and wherein an improvement in a physiological condition or a neuropsychiatric condition is achieved.

In one aspect of the invention, a method of modulating a brain activity of a person is described wherein said method comprises modulating a brain activity of a person wherein said method comprises subjecting the person to repetitive stimulating current pulses wherein the current pulse frequency is variable, and has a distribution approximating an EEG frequency distribution within a frequency range of the person, having an upper and lower frequency limit, and wherein an improvement in a physiological condition or a neuropsychiatric condition is achieved.

In another aspect, the repetitive current pulses are created through induction using rTMS. For example, the magnetic field pulses could be generated using a coil external to the head of the person. In another example, the magnetic pulses could be generated using moving permanent magnets external to the head of the person. The magnetic pulse duration could be short or long. The magnetic pulses could be sinusoidal, such that the pulse train resembles a sinusoidal wave.

In another aspect, the repetitive current pulses are created transcranially through tACS. For example, the tACS current could be generated through electrodes placed on the person's scalp. The electric pulse duration could be short or long. The pulses could be sinusoidal, such that the electric pulse train resembles a sinusoidal wave.

The variability of the administered brain stimulation may be preselected or based upon characteristics of the person's EEG. In another aspect of the invention, the frequency range is a frequency band of the person. For example, the EEG signal analyzed to determine a personalized administration of brain stimulation is based on a frequency range, such as the Alpha Band. In another aspect of the invention, the frequency band is delta band (<4 Hz), theta band (4-8 Hz), alpha band (8-13 Hz), beta band (13-30 Hz), gamma band (30-80 Hz), or Mu band (9-11 Hz). In an exemplary embodiment, the brain activity being modulated comprises one or more brain wave frequency bandwidths between 3 and 7 Hz, 8 and 13 Hz, 15 and 20 Hz, and 35 and 45 Hz

In one aspect of the invention, the EEG is recorded prior to the initiation of a treatment session. In order to reduce the burden on the person, the EEG could be recorded, for example, before the first treatment session. Alternately, the EEG could be recorded before each treatment session. In another aspect of the invention, the EEG is recorded in a time interval between current pulse trains during a treatment session, and the current administered pulse pattern is updated before each current pulse train. This updating would account for EEG changes that may occur as a result of stimulation. It is even possible to record EEG during a pulse train, and update the stimulation parameters based on that recording. In one aspect of the invention, the EEG is recorded during a current pulse train and the current pulse frequency distribution, pulse duration, pulse interval, and/or pulse intensity is updated during each current pulse train of a treatment session.

The rTMS or tACS treatment in the present invention may be used in a variety of physiological conditions. In one aspect of the invention, the physiological condition is concentration, sleep, alertness, memory, blood pressure, stress, libido, speech, motor function, physical performance, cognitive function, intelligence, height or weight. The treatment may also be used for a number of neuropsychiatric conditions. In one aspect of the invention, the neuropsychiatric condition is Autism Spectrum Disorder (ASD), Alzheimer's disease, schizophrenia, anxiety, depression, coma, Parkinson's disease, substance abuse, bipolar disorder, sleep disorder, eating disorder, tinnitus, fibromyalgia, Post Traumatic Stress Disorder (PTSD), Traumatic Brain Injry (TBI), memory impairment, pain, addiction, Obsessive Compulsive Disorders (OCD), hypertension, libido dysfunction, motor function abnormalities, small height in young children, stress, obesity, concentration/focus abnormalities, speech abnormalities, intelligence deficits, cognition abnormalities, Attention Deficit Hyperactivity Disorders (ADHD), myalgia, chronic Lyme disease, Rheumatoid Arthritis (RA), autoimmune disease, gout, diabetes, arthritis, trauma rehab, athletic performance, cognitive improvement, or stroke.

### BRIEF DESCRIPTION OF THE DRAWINGS

A better understanding of the features and advantages of the devices and methods provided will be obtained by reference to the following detailed description that sets forth illustrative embodiments and the accompanying drawings of which:

FIG. 1A shows an exemplary EEG raw signal is illustrated with superimposed wave pattern determined based on the raw signal. FIG. 1B shows an exemplary pulse wave form corresponding to the superimposed wave pattern of FIG. 1A.

FIG. 2 shows an exemplary EEG frequency distribution, which specifies the range of the frequency spectrum for the current pulses, which approximates the EEG frequency distribution.

FIG. 3 shows an exemplary EEG frequency distribution, in which the range of frequency spectrum for the current pulses is defined by the EEG spectrum crossing a threshold.

FIG. 4 shows an exemplary EEG frequency distribution, in which a Gaussian curve is fitted to the frequency distribution within a defined range. The Gaussian distribution may be used to define the frequency spectrum for the current pulses, which approximates the Gaussian distribution within the defined range.

FIG. 5A shows a frequency distribution and FIG. 5B shows a time plot of current pulses, which vary in frequency and approximate the frequency distribution within the defined range of FIG. 5A.

FIG. 6A shows the primary frequency distribution for the pulse trains in a treatment session, and the frequency distribution at the 1st higher harmonic and the 2nd higher harmonic also represented. FIG. 6B illustrates the exemplary time plot of current pulses corresponding to FIG. 6A.

FIG. 7A shows a frequency distribution, and FIG. 7B shows a time plot of current pulses, which vary in amplitude and frequency that approximate the frequency distribution within the defined range of FIG. 7A.

FIG. 8 shows a sample EEG, along with a wave pattern composed of concatenated sine waves, each of which approximates a section of the EEG, in which current pulses are generated at the peaks of the wave.

### DETAILED DESCRIPTION

While certain embodiments have been provided and described herein, it will be readily apparent to those skilled in the art that such embodiments are provided by way of example only. It should be understood that various alternatives to the embodiments described herein may be employed, and are part of the invention described herein.

Conventionally, rTMS or tACS pulses are administered at a constant pulse width, amplitude, and/or pulse frequency. However, an individual's EEG may vary over time and from one person to the next, or may be composed of a variety of individual signals that creates a distribution of parameters. Instead of simply using an average or general parameter, exemplary embodiments described herein include systems and methods for providing a variable pulse train to a patient. The variable pulse train may be determined based on the variability of one or more attributes of the patient's EEG signal. For example, the EEG signal may define a wave pattern that includes variable pulse amplitudes, variable pulse widths, and/or variable pulse frequencies.

The brain activity shown in an EEG record, even when averaged over a specific frequency range, does not generate a constant wave pattern. Instead, the EEG record will vary over time or from one person to the next. The EEG record may also comprise a distribution of signals and have variations within the signal attribute. Even after a wave pattern is extracted from the EEG raw information, the extracted wave pattern will likely be variable in wave amplitude, wave period, wave duration, and wave frequency.

Exemplary embodiments include applying a magnetic field to a patient that may be varied in amplitude, pulse duration, pulse interval, frequency, pulse train duration, and combinations thereof in response to an analyzed EEG signal, and the variability of the analyzed EEG signal of the individual patient. For example, an EEG recording may be analyzed to generate a wave pattern. The wave pattern may vary over time. The wave pattern may vary over a distribution of a detected attribute defining the EEG signal. In either case, the wave pattern may define a unique wave pattern specific to an individual patient. The unique wave pattern may also be defined by or include a variability of an attribute. The variability of an attribute may be in a wave form amplitude, wave duration, wave interval, wave frequency, or may include a distribution of a given attribute within the EEG recording. The unique wave pattern may be analyzed to optimize the administration of brain stimulation. The optimal stimulation may be administered based on the variability of the unique wave pattern. For example, an optimal stimulation may be administered at a variable pulse duration, variable pulse interval, variable pulse intensity, variable pulse frequency, and combinations thereof. Described herein are methods for the treatment of a person using rTMS or tACS with a variable pulse interval, variable pulse intensity, variable pulse frequency, variable pulse duration, and combinations thereof. Many choices exist for patterns of current pulses.

In one aspect of the invention, a method of modulating a brain activity of a person is described wherein said method comprises modulating a brain activity of a person wherein said method comprises subjecting the person to repetitive stimulating current pulses wherein the current pulse frequency, pulse duration, pulse interval, pulse intensity, pulse train duration, and combinations thereof are variable creating a variable pulse pattern. The administered variable pulse pattern may have a parameter that is determined from an EEG signal of a patient. The administered variable pulse pattern may be based on a variability of an analyzed EEG signal. The administered variable pulse pattern may be based on an EEG wave pattern including amplitude, wave duration, wave interval, frequency distribution, and combinations thereof

The administered pulse train may include a variable attribute, such as the frequency range, pulse duration, pulse interval, pulse amplitude, pulse train duration, and combinations thereof. The administered pulse train variable attribute may be preselected or based upon characteristics of the person's EEG. In another aspect of the invention, the EEG signal is based on a frequency band of the person. For example, the frequency range could be the Alpha Band, such that the analyzed EEG signal and corresponding administered pulse train is based on or approximates an activity in the Alpha Band of the person. In another aspect of the invention, the frequency band is delta band (<4 Hz), theta band (4-8 Hz), alpha band (8-13 Hz), beta band (13-30 Hz), gamma band (30-80 Hz), or Mu band (9-11 Hz). It would be possible also to approximate multiple frequency bands in one treatment session, by varying the period between current pulses so that the frequency distribution of current pulses within the two ranges approximates the EEG distribution of the person.

In another aspect, the repetitive current pulses are created through induction using rTMS. For example, the magnetic field pulses could be generated using a coil external to the head of the person. In another example, the magnetic pulses could be generated using moving permanent magnets external to the head of the person. The magnetic pulse duration could be short or long. The magnetic pulses could be sinusoidal, such that the pulse train resembles a sinusoidal wave.

In another aspect, the repetitive current pulses are created transcranially through tACS. For example, the tACS current could be generated through electrodes placed on the person's scalp. The electric pulse duration could be short or long. The pulses could be sinusoidal, such that the electric pulse train resembles a sinusoidal wave.

The applied pulse pattern administered to a person may be based on a variability within a recorded EEG signal of the person. In one aspect of the invention, the EEG signal of a patient is recorded. The EEG signal may be recorded prior to the initiation of a treatment session. In order to reduce the burden on the person, the EEG could be recorded, for example, before the first treatment session, with the EEG signal attributes or wave form from that EEG being used for all subsequent treatments. Alternately, the EEG could be recorded before each treatment session. In another aspect of the invention, the EEG is recorded in a time interval between current pulse trains during a treatment session, and the current pulse frequency distribution is updated before each current pulse train. This updating would account for EEG changes that may occur as a result of stimulation, or minor EEG variations that occur over a relatively short period of time. It is even possible to record EEG during a pulse train, and update the stimulation parameters based on that recording. In one aspect of the invention, the EEG is recorded during a current pulse train and the current pulse frequency distribution is updated during each current pulse train of a treatment session. This aspect would be difficult to implement, however, due to the significant effect that current pulses from rTMS or tACS have on the person's EEG.

The EEG data may thereafter be analyzed to determine a variability in the EEG data. For example, the analysis may extract a wave pattern from the EEG signal. In an exemplary embodiment, the analysis may be a wavelet transform. The wavelet may also be generated by curve-fitting a prespecified parameterized wavelet and using an optimization routine. The wavelet may also be generated by concatenating a series of sub-wavelets, each of which are parameterized to approximate the EEG signal in a specified range. A wave pattern may also be generated by parametric curve fitting. Any fitted wave form that may provide for oscillatory behavior within the EEG signal may be used, such as sinusoidal, parametric polynomical, etc. A wave pattern may be generated from the raw amplitude signal of an average from a desired frequency band taken over time.

The wave pattern may then be used to determine a variation in an attribute to vary an attribute of the pulse pattern of the stimulation supplied to a patient. For example, a wave pattern may include sequential peaks and troughs when represented as an amplitude over time for a given frequency band of the patient's EEG. A period between sequential peaks or between sequential troughs or between adjacent peak to trough may change over time. The amplitude variation between sequential minimum and maximum peaks may also change over time. A wave pattern from a patient's EEG signal may be used to determine a pulse pattern. The pulse pattern may include a pulse at a variable period and/or variable intensity. For example, the pulse pattern may align with a maximum peak of the wave pattern generated from the patient's EEG signal. The pulse pattern may comprise a variable pulse interval based on an interval of the wave pattern, such as a time interval between sequential maximum peaks in the wave pattern. The pulse pattern may align with a minimum peak (trough) of the wave pattern generated form the patient's EEG signal. The pulse pattern may comprise a variable intensity based on an amplitude variation of the wave pattern. For example, a pulse intensity may be proportional to or be based on an amplitude difference from sequential peak to trough or trough to peak of the wave pattern from a patient's EEG signal.

In an exemplary embodiment, a pulse pattern of magnetic pulses may be administered to a patient in which the rTMS pulse occurs at a time corresponding to a peak of the wave pattern generated from the patient's EEG signal. The timing between pulses of the variable pulse pattern, the pulse interval, may be equal to, based on, or proportional to a time duration between peaks, troughs, peak to trough, or trough to peak of the wave pattern generated from the patient's EEG signal. In an exemplary embodiment, a variable pulse pattern administered to a patient may therefore approximate a pulse interval between sequential pulses at approximately the same duration as a time interval between sequential peaks of the wave pattern. For brain wave activity in the alpha band, the pulse pattern may include a variable pulse interval between 75 milliseconds and 125 milliseconds.

The applied pulse pattern administered to a person may be based on a variability within a recorded EEG signal of the person. Methods described herein may therefore include obtaining an EEG data set from a patient. The EEG data set may thereafter be analyzed to generate a wave pattern. The EEG data set may be analyzed with a wavelet transform to generate a wave pattern. The EEG data set may be analyzed to generate a wave pattern in the form of a distribution of a variable attribute of the EEG signal. The wave pattern may be used to determine a variable attribute used to program the pulse pattern to be administered to the patient. The variable pulses are administered to a patient based on the pulse pattern. The pulse pattern may comprise a variable pulse interval. The variable pulse pattern may be used to program an apparatus as described herein to administer pulses as variable pulse intervals to a patient.

In an exemplary embodiment, the peak power or intensity delivered to a patient is below the patient's motor threshold. For example, the intensity delivered to a patient may be between 40 and 90 percent of the patient's motor threshold. In an exemplary embodiment, the intensity of the pulse pattern may be proportional to the wave pattern generated from the EEG data. The proportionality may be set between a desired intensity range, such as that based on the patient's motor threshold and on the patient's comfort level.

The EEG data may thereafter to analyzed to determine a variability in the EEG data. For example, the analysis may extract a wave pattern from the EEG signal, where the wave pattern is on a distribution of a variable within the EEG data. Brain activity as shown in EEG recordings, does not occur at a single frequency, but instead is composed of aggregate neuronal firings at a variety of frequencies, such that the frequency spectrum consists of the summations of the rhythmic firings of a large number of neurons around average intrinsic frequencies. This causes the frequency distribution to be somewhat bell-shaped. A single neuron in the brain may fire at a specific frequency. However, that frequency may be different from other neurons in other parts of the brain. It is the accumulation of neuronal firings that creates a recordable EEG, with the summation of neuronal firing frequencies creating the EEG frequency spectrum. Therefore, the IAF of a person may be different depending on which portion of the brain is being recorded. If one were able to electrically isolate regions of the brain, each region would likely provide an EEG waveform with an intrinsic frequency different from other regions, even those in close physical proximity to the region of interest.

The applied wave pattern administered to a person may be based on the frequency spectrum of a recorded EEG of the person. The firing of neurons in a region of the brain exhibit resonant behavior, with a particular intrinsic frequency or frequencies. In order to affect neurons in that region, therefore, it may be desirable to provide current pulses at a frequency that matches, or is a harmonic of, the region's intrinsic frequency. The intrinsic frequency of neighboring regions may vary, such that the frequency distribution of neuronal firing resembles a bell-shape, with a large proportion of neuronal tissue firing at the overall intrinsic frequency, and smaller proportion of tissue firing at frequencies that are farther from the overall intrinsic frequency. One could represent the EEG frequency distribution as a histogram of recorded neuronal firing frequencies sampled at one location on the scalp. It is optimal, though not required, to provide stimulation at or near the location on the scalp where the EEG is sampled. One could sample EEG at multiple locations on the scalp, to compile a collection of EEG frequency distributions across the brain, and select one or more of the EEG scalp locations, using the frequency distribution at one of the locations, or combining the frequency distribution from multiple locations, and use the resulting EEG frequency distribution to calculate the frequency distribution of current pulses.

In order to affect all regions of the brain, the frequency of magnetic pulses may be varied, so that the frequency spectrum of the magnetic pulses over the entire treatment session approximate a frequency distribution of the EEG within a specified range.

In one aspect of the invention, a method of modulating a brain activity of a person is described wherein the method comprises modulating a brain activity of a person wherein said method comprises subjecting the person to repetitive stimulating current pulses wherein the current pulse frequency is variable, and has a distribution approximating an EEG frequency distribution within a frequency range of the person, having an upper and lower frequency limit, and wherein an improvement in a physiological condition or a neuropsychiatric condition is achieved.

Many choices exist for determining a pulse patterns or current pulses so that a frequency spectrum of current pulses approximates the EEG frequency distribution within the specified range. For example, the pulses could be generated randomly, in which a random number is chosen between the period for the highest frequency in the specified range and the period for the lowest frequency in the specified range, and the period between each pulse and the next could vary based on this random number. In this case, the histogram of the reciprocal of each period over the treatment session would resemble the frequency distribution of the EEG of the person. In another example, the pulses could vary by generating consecutive pulse trains, where the duration of each pulse train is chosen so that the histogram of pulse frequencies resembles the frequency distribution of the EEG of the person. In another example, the simplest way to implement the frequency variation is to sweep the frequency within the range from low to high or high to low, with some frequency step, where the duration of the pulse train at each frequency is proportional to the energy of the frequency distribution of the person's EEG at that frequency. In another example, the pulse trains are randomly assigned variable pulse frequencies with tabulated occurrence such that the distribution of all pulse frequencies delivered upon completion of delivery resembles the frequency distribution of the EEG of the person receiving the treatment.

The administered pulse train may include a variable attribute, such as the frequency range, pulse duration, pulse width, pulse interval, pulse amplitude, pulse train duration, and combinations thereof. The administered pulse train variable attribute may be preselected or based upon characteristics of the person's EEG. In another aspect of the invention, variable pulse train is based on a frequency range and may be a frequency band of the person. For example, the frequency range could be the Alpha Band, such that the distribution of current pulse frequency approximates the activity in the Alpha Band of the person. In another aspect of the invention, the frequency band is delta band (<4 Hz), theta band (4-8 Hz), alpha band (8-13 Hz), beta band (13-30 Hz), gamma band (30-80 Hz), or Mu band (9-11 Hz). It would be possible also to approximate multiple frequency bands in one treatment session, by varying the period between current pulses so that the frequency distribution of current pulses within the two ranges approximates the EEG distribution of the person.

In another aspect of the invention, the frequency range is a predetermined range around an intrinsic EEG frequency of an EEG band. This range could be equidistant around the intrinsic EEG frequency. For example, the frequency range could be from the person's IAF−2.0 Hz to IAF+2.0 Hz. The choice of intrinsic EEG frequency could vary depending on the type of therapy being delivered. In another aspect of the invention, the intrinsic EEG frequency is a delta frequency, theta frequency, alpha frequency, beta frequency, gamma frequency, or Mu frequency. This aspect may be preferred to target EEG frequencies that are close to the intrinsic frequency. For example, the alpha range (8-13 Hz) covers 5 Hz. If the person's IAF is 12.5 Hz, then the neuronal activity in the 8-9 Hz range may not significantly affect the overall resonance, whereas the lower range of the beta band (13-14 Hz) may play a part in the brain resonance, since it is close to the IAF. Therefore, specifying a range on either side of the intrinsic frequency may bring more resonant neuronal activity into play. The range does not have to be equidistant around the intrinsic EEG frequency. For example, the frequency range could be from the person's IAF−2.0 Hz to IAF+1.0 Hz.

Instead of an arbitrary range surrounding an intrinsic EEG frequency, the range could be based on the distribution itself In one aspect of the invention, the frequency range is a range about an intrinsic EEG frequency of an EEG band with limits set to frequency values where the EEG frequency distribution amplitude is a predetermined percent of the EEG frequency distribution amplitude at the intrinsic frequency. For example, the range could extend to a point where the amplitude of the frequency distribution is 30% of the maximum value, or the value at the intrinsic frequency. This aspect of the method would allow the minimum range that maximizes the energy of the frequency distribution. However, this aspect is also susceptible to variations in the frequency spectrum of the EEG, since the frequency spectrum does not decrease monotonically on either side of the intrinsic EEG frequency.

Instead of exactly matching the frequency distribution within a range, it would be possible instead to approximate the frequency distribution with a known distribution that can be parameterized. By doing this, implementation of the frequency variation may be simpler. In one aspect of the invention, the magnetic pulse frequency's distribution is Gaussian, with a mean and standard deviation that approximates the mean and standard deviation of the EEG frequency distribution. In another aspect of the invention, the magnetic pulse frequency's distribution is uniform, with a mean and standard deviation that approximates the mean and standard deviation of the EEG frequency distribution. Other distributions are possible as well, including Poisson distribution, Bernoulli distribution, Binomial distribution, Skellam distribution, Chi-squared distribution, or Gamma distribution.

The rTMS or tACS treatment in the present invention may be used in a variety of physiological conditions. In one aspect of the invention, the physiological condition is concentration, sleep, alertness, memory, blood pressure, stress, libido, speech, motor function, physical performance, cognitive function, intelligence, height or weight. The treatment may also be used for a number of neuropsychiatric conditions. In one aspect of the invention, the neuropsychiatric condition is Autism Spectrum Disorder (ASD), Alzheimer's disease, schizophrenia, anxiety, depression, coma, Parkinson's disease, substance abuse, bipolar disorder, sleep disorder, eating disorder, tinnitus, fibromyalgia, Post Traumatic Stress Disorder (PTSD), Traumatic Brain Injury (TBI), memory impairment, pain, addiction, Obsessive Compulsive Disorders (OCD), hypertension, libido dysfunction, motor function abnormalities, small height in young children, stress, obesity, concentration/focus abnormalities, speech abnormalities, intelligence deficits, cognition abnormalities, Attention Deficit Hyperactivity Disorders (ADHD), myalgia, chronic Lyme disease, Rheumatoid Arthritis (RA), autoimmune disease, gout, diabetes, arthritis, trauma rehab, athletic performance, cognitive improvement, or stroke.

Exemplary embodiments include a system and apparatus for generating a pulse train to a patient having a variable attribute. The apparatus may include components for generating repetitive magnetic or current pulses. Exemplary embodiments described herein may include an apparatus for generating repetitive current pulses. The apparatus may be configured to generate the repetitive current pulses through induction using rTMS. For example, the magnetic field pulses could be generated using a coil external to the head of the person. In another example, the magnetic pulses could be generated using moving permanent magnets external to the head of the person. The magnetic pulse duration could be short or long. The magnetic pulses could be sinusoidal, such that the pulse train resembles a sinusoidal wave. The apparatus may also be configured to generate the repetitive current pulses transcranially through tACS. For example, the tACS current could be generated through electrodes placed on the patient's scalp. The electric pulse duration could be short or long. The pulses could be sinusoidal, such that the electric pulse train resembles a sinusoidal wave.

The apparatus may include electrodes for detecting an EEG signal from a patient. The apparatus may be configured to receive an EEG signal of a patient.

The apparatus may include processor and/or memory to analyze the EEG signal. In an exemplary embodiment the apparatus may be configured to communicate over a network to a remote processor and/or memory to analyze the EEG signal. The processor(s) and/or memory may therefore be contained within a common housing of the apparatus or may be remote from the leads for detecting the EEG and/or from the components for generating a magnetic or current pulse train.

In an exemplary embodiment, the memory comprises non-transitory machine-readable instructions that, when executed by the processor(s), is configured to perform the functions described herein. For example, the instructions may include software for determining a variability of the EEG signal. The variability may be in a frequency distribution of the EEG signal. The variability of an attribute may be in a wave form amplitude, wave duration, wave interval, wave frequency, or may include a distribution of a given attribute within the EEG recording. The instructions may be configured to approximate a wave form from the EEG signal having variability of an attribute. The approximate wave form may be used to obtain the attribute and/or value of the variability of the attribute. The instructions may also be configured to control the generation of the magnetic or current pulses to be administered to the patient. The instructions may be configured to administer the magnetic or current pulses based on the variability of the attribute.

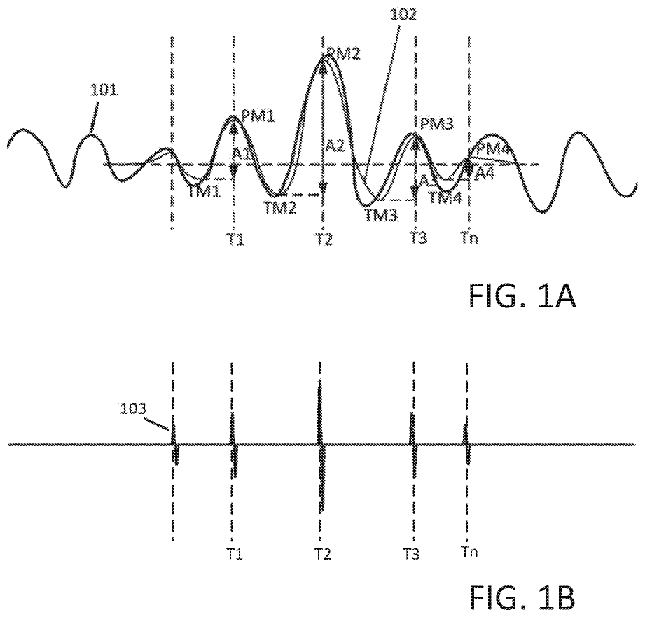

FIG. 1 shows an exemplary EEG signal (101) of an average amplitude over time averaged from a frequency band of a patient. The frequency band illustrated corresponds to the alpha band of approximately 8-13 Hz, but other ranges may also be used. The EEG signal (101) may be analyzed to extract a wave form (102) from the EEG raw data signal. The wave form (102) may define a cyclical wave form or a burst wave form with one or more variable attributes. The wave form, as illustrated, include sequential peaks indicating a peak maximum amplitude (PM) and sequential trough minimum amplitudes (TM). Each peak occurs at a time (T). The difference between sequential peaks (PM) may be used to determine a period (Tn+1−Tn). A peak maximum occurs between a peak minimum, such that a waveform may be created from the oscillations between minimums and maximums. An amplitude (A) may be defined as the amplitude difference between sequential troughs (TM) and peaks (PM), or vice versa. For reference, sequential peaks and troughs and corresponding amplitudes and timing are indicated with a sequential numerical value for reference only. The sequence or includes of a specific number is not required, and may include any sequence greater than 0 and up to n. A pulse pattern to administer magnetic pulses to a patient may be generated from the attributes of the wave form, such as the waveform period, amplitude, peak timing, and combinations thereof. For example, the pulse pattern may start with a time corresponding to an anticipated time of a peak maximum of the wave form. The pulse pattern may include a variable period based on a period or corresponding to a period between adjacent peak maximums of the wave form. The pulse pattern may include a variable intensity approximate to, proportional to, or otherwise related to the amplitude of the wave form. An exemplary pulse wave form (103) is provided, showing a burst stimulation composed of five pulses, with timing defined by the peak locations of the wave form (102), and pulse amplitude defined by the amplitude of the peaks of the wave form. The pulse train could be repeated in order to provide a longer burst or continuous pulse stimulation to the patient. The pulse amplitude may be constant or variable as shown. Also, the average time interval between peaks may be determined and used to define a frequency value for the pulses.

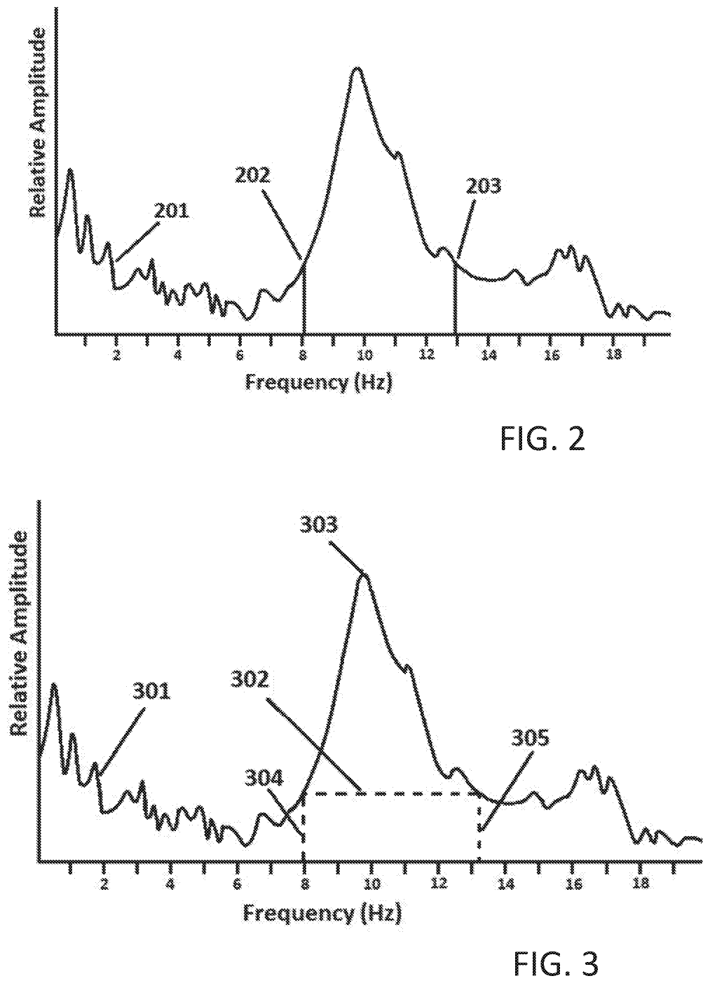

FIG. 2 shows an exemplary EEG frequency distribution (201) for a person. In this, the frequency distribution within a range defined by a low (202) and a high (203) frequency has been chosen. The current pulse frequency distribution may be chosen to approximate that frequency distribution between the low and high range.

FIG. 3 shows an exemplary EEG frequency distribution (301) for a person, with an intrinsic EEG frequency (303). An amplitude value (302) is chosen, with the low (304) and high (305) frequency values being set to the point where the value crosses the frequency spectrum. The low and high frequency values define a frequency range, and the current pulse frequency distribution may be chosen to approximate that frequency distribution between the low and high values.

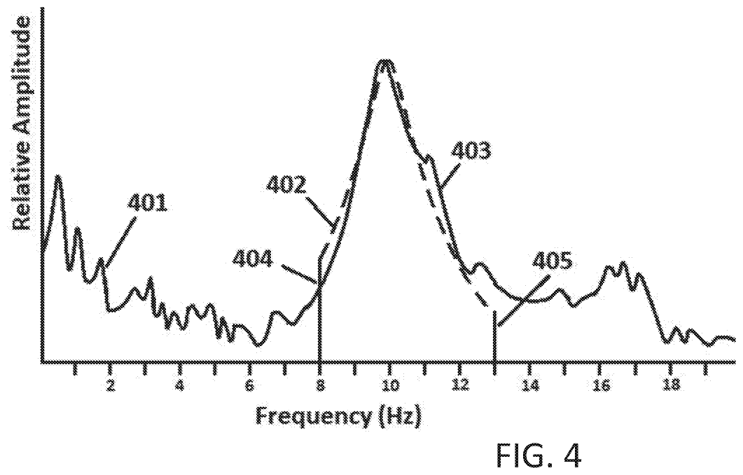

FIG. 4 shows an exemplary EEG frequency distribution (401), where a Gaussian curve (402) has been optimized to approximate the frequency distribution (403) within a range specified by a low (404) and high (405) frequency. The current pulse frequency distribution may be chosen to approximate the curve in the range between the low and high frequency values.

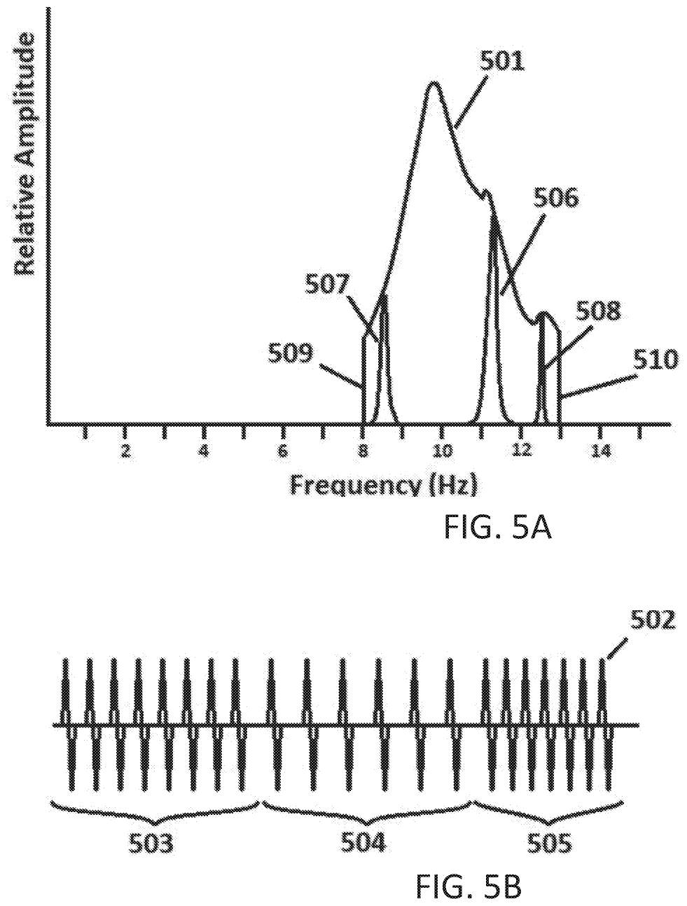

FIG. 5A shows a frequency distribution (501) that falls in a range between a low (509) and high (510) frequency. FIG. 5B shows a time series of biphasic magnetic pulses (502), where the pulse train duration at any particular pulse frequency will contribute to the frequency distribution of the entire treatment session. In this example, the low frequency pulses (504) correspond to a low frequency spike in the frequency spectrum (507). The medium frequency pulses (503) correspond to a medium frequency spike (506) in the frequency spectrum. The high frequency pulses (505) correspond to a high frequency spike (508) in the frequency spectrum. As more pulse trains at various frequencies are included in the treatment session, more spikes contribute to the frequency spectrum of the treatment session, and the duration of the pulse trains at each frequency are chosen so that the frequency spectrum based on the sum of all the spikes approximates the EEG frequency distribution for the person.

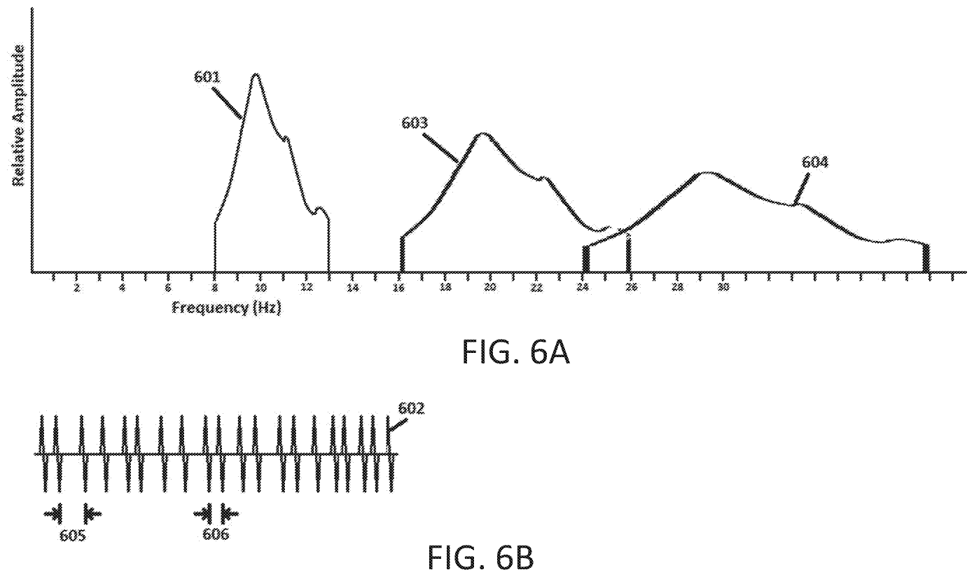

A pulse train generates frequency spikes at harmonics of the pulse frequency. Therefore, the overall frequency distribution of the treatment session will actually extend beyond the specified range. FIG. 6A shows the primary frequency distribution (601) for the pulse trains in a treatment session, and the frequency distribution at the 1st higher harmonic (603) and the 2nd higher harmonic (604) are also represented. FIG. 6B shows a time series of biphasic magnetic pulses (602) having variable pulse intervals (606). It may be possible to gain additional benefit from higher frequency components. The effect of higher frequency components may be reduced by increasing the pulse length. For example, a standard rTMS pulse is approximately 200 usec. By increasing to 300 usec, the effect of the harmonics is less.

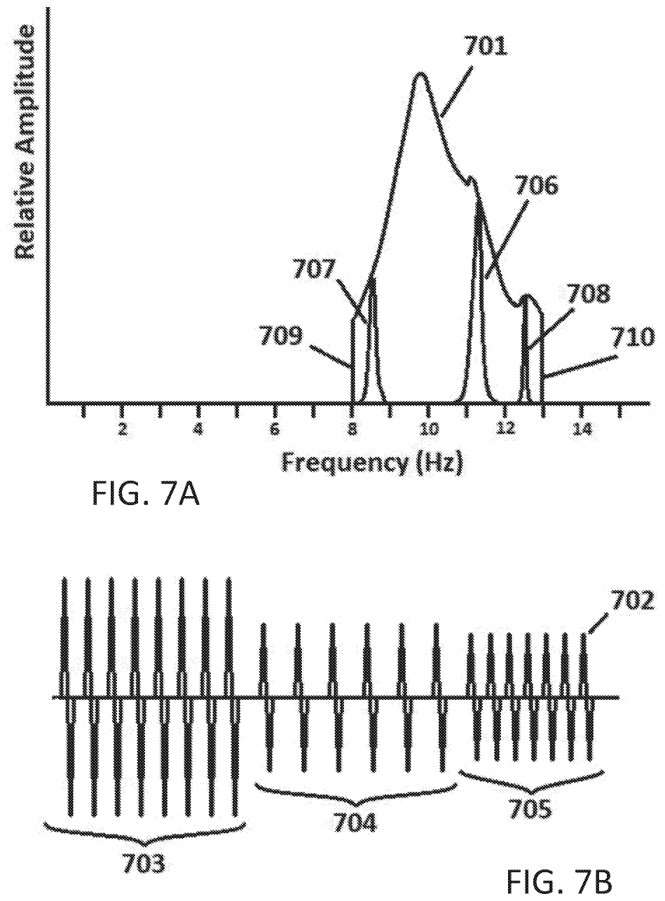

In FIG. 5B, the frequency spectrum of pulses was created by altering the duration or number of pulses at various pulse frequencies, while keeping the pulse amplitude constant. A similar outcome may be achieved by varying the pulse amplitudes, either while varying the duration or number of pulses or keeping the duration or number of pulses at various pulse frequencies constant. FIG. 7a shows a frequency distribution (701) that falls in a range between a low (709) and high (710) frequency. FIG. 7B shows a time series of biphasic magnetic pulses (702), where the pulse train amplitude at any particular pulse frequency will contribute to the frequency distribution of the entire treatment session. In this example, the low frequency pulses (704) correspond to a low frequency spike in the frequency spectrum (707). The medium frequency pulses (703) correspond to a medium frequency spike (706) in the frequency spectrum. The high frequency pulses (705) correspond to a high frequency spike (708) in the frequency spectrum. As more pulse trains at various frequencies are included in the treatment session, more spikes contribute to the frequency spectrum of the treatment session, and the pulse amplitude of the pulse trains at each frequency are chosen so that the frequency spectrum based on the sum of all the spikes approximates the EEG frequency distribution for the person.

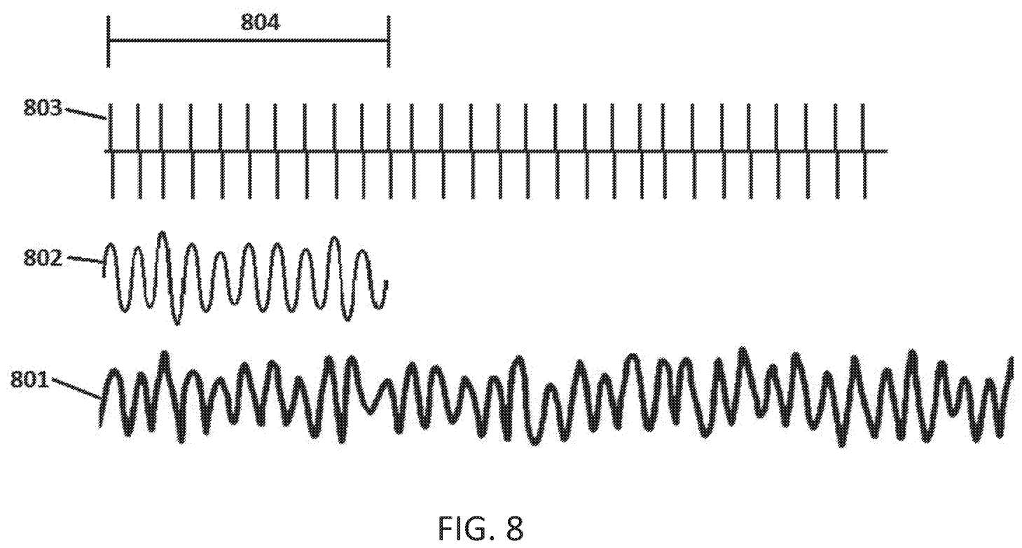

FIG. 8 shows an exemplary EEG (801), along with a wave form that is composed of concatenated sine waves (802), each of which may have a different amplitude and period. Each sine wave approximates a single period of the EEG. When the sine waves are concatenated together, they form a wave form which can be used to specify pulses (803), which are delivered to the patient. In the figure, a series of 10 sine waves are concatenated together within a range (804). In this example, the pulses occur at the peaks of the waveform. By administering pulses at the peaks of the waveform that approximates the EEG, the frequency spectrum of the pulses will approximate the frequency spectrum of the EEG within a specific frequency band. The pulses may be administered in a burst, only covering the range where the wave form has been created, or the pulses may repeat the pattern of pulses generated by the wave form, in order to provide continuous or long-term stimulation.

The description herein is generally in terms of treatment of a person. However, the disclosure is not so limited but may be applicable to any subject. “Patient” and “subject” are synonyms, and are used interchangeably. As used herein, they mean any animal (e.g. a mammal on which the inventions described herein may be practiced. Neither the term “subject” nor the term “patient” is limited to an animal under the care of a physician.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above descriptions of illustrated embodiments of the methods or devices are not intended to be exhaustive or to be limited to the precise form disclosed. While specific embodiments of, and examples for, the methods or devices are described herein for illustrative purposes, various equivalent modifications are possible within the scope of the methods, or devices, as those skilled in the relevant art will recognize. The teachings of the methods or devices provided herein can be applied to other processing methods or devices, not only for the methods or devices described.

The elements and acts of the various embodiments described can be combined to provide further embodiments. These and other changes can be made to the device in light of the above detailed description.

In general, in the following claims, the terms used should not be construed to limit the methods or devices to the specific embodiments disclosed in the specification and the claims, but should be construed to include all processing devices that operate under the claims. Accordingly, the methods and devices are not limited by the disclosure, but instead the scopes of the methods or devices are to be determined entirely by the claims.

While certain aspects of the methods or devices are presented below in certain claim forms, the inventor contemplates the various aspects of the methods or devices in any number of claim forms. Accordingly, the inventors reserve the right to add additional claims after filing the application to pursue such additional claim forms for other aspects of the methods or devices.

What is claimed is:

1. A method of modulating a brain activity of a mammal which comprises subjecting the mammal to repetitive transcranial magnetic stimulation (rTMS) with variable pulse intervals for a time sufficient to modulate the brain activity.

2. The method of claim 1, wherein the brain activity being modulated comprises one or more brain wave frequency bandwidths between 3 and 7 Hz, 8 and 13 Hz, 15 and 20 Hz, and 35 and 45 Hz.

3. The method of claim 2, wherein the brain activity being modulated is a brain wave frequency bandwidth between 8 and 13 Hz.

4. The method of claim 1, wherein the variable pulse intervals are derived from the mammal's EEG signal extracted by wavelet analysis.

5. The method of claim 4, wherein the brain activity being modulated comprises one or more brain wave frequency bandwidths between 3 and 7 Hz, 8 and 13 Hz, 15 and 20 Hz, and 35 and 45 Hz.

6. The method of claim 5, wherein the brain activity being modulated is a brain wave band between 8 and 13 Hz,

7. A method of treating PTSD in a human patient which comprises:
a. subjecting the patient to an EEG to create an EEG data set;
b. analyzing the EEG data set with a wavelet transform resulting in an EEG signal pattern;
c. using the EEG signal pattern to program an rTMS apparatus to deliver electromagnetic pulses having variable pulse intervals; and
d. subjecting the patient to repetitive transcranial magnetic stimulation (rTMS) from said programmed rTMS apparatus delivering electromagnetic pulses having variable pulse intervals derived from the wavelet transform.

8. A method of treating autism spectrum disorder (ASD) in a human patient which comprises:
a. subjecting the patient to an EEG to create an EEG data set;
b. analyzing the EEG data set with an EEG signal transform resulting in an EEG signal pattern;
c. using the EEG signal pattern to program an rTMS apparatus to deliver electromagnetic pulses having variable pulse intervals; and
d. subjecting the patient to repetitive transcranial magnetic stimulation (rTMS) from said programmed rTMS apparatus delivering electromagnetic pulses having variable pulse intervals derived from the wavelet transform.

9. A method of treating Alzheimer's disease in a human patient which comprises:
a. subjecting the patient to an EEG to create an EEG data set;
b. analyzing the EEG data set with an EEG signal transform resulting in an EEG signal pattern;
c. using the EEG signal pattern to program an rTMS apparatus to deliver electromagnetic pulses having variable pulse intervals; and
d. subjecting the patient to repetitive transcranial magnetic stimulation (rTMS) from said programmed rTMS apparatus delivering electromagnetic pulses having variable pulse intervals derived from the wavelet transform.

10. The method of claim 1, wherein the rTMS stimulation is below the motor threshold of the mammal.

11. The method of claim 10, wherein the rTMS delivered is 40-90 percent of the motor threshold of the mammal.

12. Use of a repetitive Transcranial Magnetic Stimulation (rTMS) apparatus made to generate and deliver rTMS pulses at variable pulse intervals for the treatment of Post Traumatic Stress Disorder (PTSD); Autism Spectrum Disorder (ASD), and Alzheimer's Disease (AD).

13. The rTMS apparatus of claim 12, wherein the rTMS apparatus is programmed to deliver electromagnetic pulses at variable pulse intervals derived from a wavelet transform.

14. The rTMS apparatus of claim 13, used for the treatment of Post Traumatic Stress Disorder (PTSD), Autism Spectrum Disorder (ASD), Alzheimer's Disease (AD), Traumatic Brain Injry (TBI), memory impairment, depression, pain, addiction, Obsessive Compulsive Disorders (OCD), anxiety, Parkinson's Disease (PD), hypertension, libido dysfunction, motor function abnormalities, small height in young children, stress, obesity, sleep disorders, eating disorders, concentration/focus abnormalities, speech abnormalities, intelligence deficits, cognition abnormalities, Attention Deficit Hyperactivity Disorders (ADHD), schizophrenia, coma, bipolar disorders, tinnitus, fibromyalgia, chronic Lyme disease, Rheumatoid Arthritis (RA), autoimmune disease, gout, diabetes, arthritis, trauma rehab, athletic performance, cognitive improvement, and stroke.

15. An improved rTMS apparatus, wherein the improvement comprises programming the rTMS apparatus to deliver rTMS pulses at variable pulse intervals.

16. The improved rTMS apparatus of claim 15 wherein the variable pulse intervals are derived from a wavelet transform.

17. An rTMS apparatus that generates magnetic pulses which comprises a program in the apparatus that generates magnetic pulses at variable pulse intervals.

18. The rTMS apparatus of claim 17, wherein the variable pulse intervals are derived from a wavelet transform.
